(19) United States
(12) Patent Application Publication
(10) Pub. No.: US 2012/0164252 A1
(43) Pub. Date: Jun. 28, 2012

(54) SUPPLEMENT COMPRISING BLACKCURRANTS OR BOYSENBERRIES

(75) Inventors: Yasuhiro Kano, Sakyo-ku (JP); John Gibb, Hope (NZ); Bryce Lukes, Richmond (NZ)

(73) Assignee: GIBB HOLDINGS (NELSON) LIMITED, NELSON (NZ)

(21) Appl. No.: 13/375,107

(22) PCT Filed: May 28, 2010
(86) PCT No.: PCT/NZ10/00100

§ 371 Date: Mar. 16, 2012

(30) Foreign Application Priority Data
May 29, 2009 (NZ) 577357

(51) Int. Cl. A61K 36/73; A61P 37/00; A61K 36/185; A61P 3/00; A23K 1/16; A23K 1/18

(52) U.S. Cl. 424/765; 426/655; 426/2; 426/635; 424/777

(57) ABSTRACT

The present invention relates to a dietary supplement comprising one or more of processed whole blackcurrants, processed whole boysenberries, one or more natural component extracted from blackcurrants, and one or more natural component extracted from boysenberries. The dietary supplement is particularly, but not exclusively, suitable for animals.

### FIELD

The present invention relates to a dietary supplement, particularly but not exclusively, a dietary supplement suitable for animals.

### BACKGROUND

Muscle activity brings about a number of metabolic and physiological changes in the body, a number of which may have a negative effect on endurance, athletic performance and the ability to recover quickly following exercise.

For example, intense exercise can result in increased levels of circulating lactic acid in the blood stream which is a significant factor in exercise associated fatigue. Until the lactic acid is cleared from the blood stream the muscles cannot work effectively.

In addition, muscle inflammation and damage can occur during exercise resulting in pain and further fatigue. Due to the degree of associated muscle damage following intense exercise, it is often necessary to have training and competition down-time, that may be days to weeks in length, while injured tissues recover before high level training can recommence.

An elevated heart rate is associated with exercise. It is considered beneficial for the heart rate to resume to resting level as soon as possible after exercise, so that it is not put under any unnecessary stress. A delayed fall in heart rate after exercise may place unnecessary stress on the heart and body, increase muscle damage, and delay recovery.

A number of products including pharmaceuticals, foods, and dietary supplements have been suggested to be of benefit in dampening some of the negative metabolic and physiologic responses to exercise, with the possibility of enhancing physical performance. However, there are often problems associated with such products. For example, they may have their own side effects (including gastric distress, for example), there may be difficulties in consuming enough quantities to gain a desired affect, formulation issues may reduce or inhibit the bioavailability of active ingredients, and the products may not be palatable. Particularly in the case of supplements for use with animals, formulation of the product to ensure palatability as well as delivery of the desired amount or concentration of active ingredients can be difficult. Further, whilst existing products may address a specific metabolic or physiological side effect of exercise, performance may still be affected by one or more of the other side effects.

It has been suggested that dietary antioxidants may limit exercise induced oxidative stress and modulate inflammatory events in humans, to provide positive benefits during short periods of exercise. However, the affects of antioxidants during extended periods of exercise is not clear.

Birds raised for meat or egg production are prone to stress related conditions including depressed immune system response, lethargy, poor appetite and rejection of fluids that sometimes results in death, particularly in high temperature and/or high humidity conditions.

Bird egg and meat production is also often hindered by the impact of stress on the birds from confinement, temperature fluctuations, humidity fluctuations and diet deficits. The observed negative impacts include increased disease mortality, loss of disease resistance, reduction in egg laying rates, deterioration in egg shell integrity and colour in the case of brown or coloured eggs, reduction in egg-laying lifespan, loss of moisture from the meat, and toughening of muscle meat.

Animals for meat production are generally transported from a farm to a slaughter house prior to their slaughter. The stress of the transporting of the animal to the slaughter house can often reduce the quality of the meat.

Plants have been used historically to treat illness and improve health. In some cases health benefits are attributed to individual compounds within a plant. However, such compounds are often contained in the plant in very small quantities. In addition, due to instability, the level of biologically active quantities of the compounds may decrease with time in storage post harvest. Further, the level of absorption of the compounds into an animal's body may be very low, or once consumed, the compounds may be denatured so they are not in a biologically active form. As a result, the amount of a plant needed to be consumed in order to gain an effective dose is often very large, and beyond the volume that an animal could realistically consume. The plant may be processed to reduce the volume and increase the concentration of desired compounds, and extracts of certain components of the plants may be prepared.

However, in doing so, a number of problems can arise. For example, valuable components of the whole plant may be lost, the shelf life of the product may suffer, the active compounds may be denatured, the taste and/or texture of the product may change such that it is no longer palatable, the ability of the compounds to be absorbed into the body may decrease, and the product may produce a number of unwanted side-effects when consumed. This can often result in a product which is less viable or less valuable than consumption of the unprocessed plant. It can be extremely difficult to adequately address such issues, which are typically competing, to provide a product which is both biologically and commercially viable.

### OBJECT

It is an object of the present invention to provide at least one dietary supplement and/or method(s) which overcomes or ameliorates at least one of the disadvantages of the prior art or at least to provide the public with a useful alternative.

### STATEMENT OF INVENTION

In one broad aspect, the present invention provides a composition comprising one or more of processed whole blackcurrants, processed whole boysenberries, one or more natural component extracted from blackcurrants, and one or more natural component extracted from boysenberries.

In one particular embodiment, the invention provides a composition comprising a mixture of processed whole blackcurrants and a blackcurrant extract.

In another particular embodiment, the composition comprises a blackcurrant extract and processed whole boysenberries.

In another embodiment, the composition comprises processed whole blackcurrants and processed whole boysenberries.

In a preferred embodiment, the processed whole blackcurrants comprise a seed-in blackcurrant puree. In a preferred embodiment, the puree is dried, more preferably freeze-dried. Preferably, the dried puree is ground, more preferably coarsely. ground.

In another embodiment, the processed whole blackcurrants comprise frozen blackcurrants. In one preferred embodiment, the frozen blackcurrants are ground, more preferably coarsely ground.

In a preferred embodiment, the blackcurrant extract is an extract of the skin, seeds and/or pulp of the blackcurrant. In one preferred embodiment, the extract is a blackcurrant pomace. In one preferred embodiment, the blackcurrant extract is dried, more preferably freeze-dried. Preferably the dried extract is ground, more preferably coarsely ground. In another preferred embodiment, the extract is a pomace which is frozen, preferably free flow.

In one embodiment, the blackcurrant extract is a blackcurrant concentrate powder.

In a preferred embodiment, the processed whole boysenberries comprise frozen boysenberries. In one preferred embodiment, the frozen boysenberries are ground, more preferably coarsely ground.

In another embodiment, the processed whole boysenberries comprise a seed-in boysenberry puree. In a preferred embodiment, the puree is dried, more preferably freeze-dried. Preferably, the dried puree is ground, more preferably coarsely ground.

In a preferred embodiment, the boysenberry extract is an extract of the skin, seeds and/or pulp of the boysenberry. In one preferred embodiment, the extract is a boysenberry pomace. In one preferred embodiment, the boysenberry extract is dried, more preferably freeze-dried. Preferably the dried extract is ground, more preferably coarsely ground. In another preferred embodiment, the extract is a pomace which is frozen, preferably free flow. In one embodiment, the boysenberry extract is a powder.

In one preferred embodiment, the composition comprises a freeze-dried blackcurrant extract powder and a freeze-dried blackcurrant puree in a ratio of from approximately 3 to 2, to approximately 1 to 24 (w/w). Preferably, the composition comprises a freeze-dried blackcurrant extract powder and a freeze-dried blackcurrant puree in a ratio of approximately 1 to 3 (w/w).

In another preferred embodiment, the composition comprises a blackcurrant concentrate powder and a seed-in blackcurrant puree powder in a ratio of from approximately 3 to 2, to approximately 24 to 1 (w/w). Preferably, the composition comprises a blackcurrant concentrate powder and seed-in blackcurrant puree powder in a ratio of from approximately 3 to 1 (w/w).

In another preferred embodiment, the composition comprises a frozen blackcurrant pomace (preferably free flow) and freeze-dried blackcurrants in a ratio of from approximately 1 to 1, to approximately 9 to 1 (w/w). Preferably, the composition comprises a frozen blackcurrant pomace and freeze-dried blackcurrants in a ratio of from approximately 8 to 1 (w/w).

In one preferred embodiment, the composition comprises a frozen blackcurrant pomace (preferably free flow) and freeze-dried boysenberries in a ratio of from approximately 1 to 1, to approximately 11 to 1 (w/w). Preferably, the composition comprises a frozen blackcurrant pomace and freeze-dried boysenberries in a ratio of from approximately 9 to 1 (w/w).

In one preferred embodiment, the composition comprises a frozen, air-dried or freeze-dried blackcurrant pomade (preferably free flow) and a freeze-dried blackcurrant extract powder in a ratio of from approximately 1 to 1, to approximately 100 to 1 (w/w). Preferably, the composition comprises an air-dried blackcurrant pomace and a freeze-dried blackcurrant extract powder in a ratio of approximately 20 to 1 (w/w).

In another preferred embodiment, the composition comprises a frozen, air-dried or freeze-dried blackcurrant pomace (preferably free flow) and frozen, freeze-dried or air-dried blackcurrants in a ratio of from approximately 1 to 1, to approximately 50 to 1 (w/w). Preferably, the composition comprises a frozen blackcurrant pomace and air-dried blackcurrants in a ratio of from approximately 10 to 1 (w/w).

In a preferred embodiment, the compositions of the invention comprise from approximately 1% to approximately 35% anthocyanins (w/w). In another preferred embodiment the compositions of the invention comprise from approximately 0.2% to approximately 1% anthocyanins (w/w).

In another preferred embodiment, a single dose of the composition comprises from approximately 1 mg to approximately 1500 mg anthocyanin(s). In one preferred embodiment, a single dose of the composition comprises from approximately 1 mg to approximately 15 mg anthocyanin(s). In one preferred embodiment a single dose of the composition comprises approximately 4.5 mg anthocyanin(s). In one preferred embodiment, a single dose of the composition comprises from approximately 800 mg to approximately 1500 mg anthocyanin(s). In one preferred embodiment a single dose of the composition comprises approximately 1000 mg anthocyanin(s).

In another broad aspect, the invention provides an animal feed comprising blackcurrants, a blackcurrant extract, boysenberries, a boysenberry extract, and/or a composition as herein before described. In one preferred embodiment, the animal feed has an anthocyanin content per kg of feed of from approximately 2,000 mg to approximately 60,000 mg of anthocyanin(s). In one preferred embodiment, the animal feed has an anthocyanin content per kg of feed of 50,000 mg of anthocyanin(s). In one preferred embodiment, the animal feed has an anthocyanin content per kg of feed of 4,000 mg of anthocyanin(s).

In another broad aspect, the invention provides a method for preventing or minimising lactic acid production and/or build up in an animal during exercise, the method comprising administering to the animal blackcurrants, a blackcurrant extract, boysenberries, a boysenberry extract, and/or a composition as herein before described.

In another broad aspect, the invention provides a method for preventing or minimising exercise-induced muscle damage and/or fatigue in an animal, the method comprising administering to the animal blackcurrants, a blackcurrant extract, boysenberries, a boysenberry extract, and/or a composition as herein before described.

In another broad aspect, the invention provides a method for improving or supporting recovery of an animal following exercise, the method comprising administering to the animal blackcurrants, a blackcurrant extract, boysenberries, a boysenberry extract, and/or a composition as herein before described.

In another broad aspect, the invention provides a method for reducing heart rate recovery time in an animal following exercise, the method comprising administering to the animal blackcurrants, a blackcurrant extract, boysenberries, a boysenberry extract, and/or a composition as herein before described.

In another broad aspect, the invention provides a method for improving stamina and/or endurance in an animal, the method comprising administering to the animal blackcurrants, a blackcurrant extract, boysenberries, a boysenberry extract, and/or a composition as herein before described.

Preferably the animal is a horse, a camel, a bovine animal, or a dog.

In another broad aspect, the invention provides a method for treating and/or preventing a vascular disease in an animal, the method comprising administering to the animal blackcurrants, a blackcurrant extract, boysenberries, a boysenberry extract, and/or a composition as herein before described. Preferably, the vascular disease is laminitis. Preferably the animal is a hoofed animal.

In another broad aspect, the invention provides a method for improving taste, tenderness and/or colour in meat in an animal, the method comprising administering to the animal blackcurrants, a blackcurrant extract, boysenberries, a boysenberry extract, and/or a composition as herein before described. Preferably the animal is a bovine animal or a bird.

In another broad aspect, the invention provides a method for improving or supporting health of an animal, enhancing or supporting immune system response and/or function, reducing disease mortality, reducing the dependence on antibiotics, assisting or supporting stress recovery, reducing stress impact and/or mortality in confined populations (particularly, for example, in cases of overpopulation, high air temperatures and/or high air humidity), reducing stress impact on meat quality, improving moisture content and tenderness of meat, and/or eliminating unpleasant odours in meat, fat and skin in an animal, the method comprising administering to the animal blackcurrants, a blackcurrant extract, boysenberries, a boysenberry extract, and/or a composition as herein before described.

Preferably the animal is a bird or a bovine animal.

In another broad aspect, the invention provides a method for improving or supporting egg quality and/or productive egg laying life span, preventing deterioration in egg shell integrity, enhancing egg-laying performance, egg quality and colour, egg white and yolk texture, nutritional value and/or odour, and/or increasing polyphenol content in an egg in a bird, the method comprising administering to the bird blackcurrants, a blackcurrant extract, boysenberries, a boysenberry extract, and/or a compositions as herein before described.

Preferably, the method is for improving one or more of shell strength, shell integrity, firmness of egg white, consistency of egg white, vitamin A content, the number of A grade eggs laid and maintaining egg shell colour during the laying life of the bird.

In one particular embodiment, the blackcurrants, blackcurrant extract, boysenberries, boysenberry extract, and/or the composition are administered to the animal in sufficient quantity to provide a dose of approximately 0.5 to approximately 5 mg of anthocyanin(s) per kg of the animal per day. In a preferred embodiment, the dose is approximately 1.5 mg/kg/day.

In another broad aspect, the invention provides the use of blackcurrants, a blackcurrant extract, boysenberries, and/or a boysenberry extract, in the manufacture of a medicament or dietary supplement for preventing or minimising lactic acid production and/or build up during exercise in an animal.

In another broad aspect, the invention provides the use of blackcurrants a blackcurrant extract, boysenberries, and/or a boysenberry extract in the manufacture of a medicament or dietary supplement for preventing or minimising exercise-induced muscle damage and/or fatigue in an animal.

In another broad aspect, the invention provides the use of blackcurrants, a blackcurrant extract, boysenberries, and/or a boysenberry extract in the manufacture of a medicament or dietary supplement for improving or supporting recovery following exercise in an animal.

In another broad aspect, the invention provides the use of blackcurrants, a blackcurrant extract, boysenberries, and/or a boysenberry extract in the manufacture of a medicament or dietary supplement for reducing heart rate recovery time following exercise in an animal.

In another broad aspect, the invention provides the use of blackcurrants, a blackcurrant extract, boysenberries, and/or a boysenberry extract in the manufacture of a medicament or dietary supplement for improving stamina and/or endurance in an animal.

Preferably the animal is a horse, a camel, a bovine animal or a dog.

In another broad aspect, the invention provides the use of blackcurrants, a blackcurrant extract, boysenberries, and/or a boysenberry extract in the manufacture of a medicament or dietary supplement for treating and/or preventing a vascular disease in an animal. Preferably, the vascular disease is laminitis. Preferably the animal is a hoofed animal.

In another broad aspect, the invention provides the use of blackcurrants, a blackcurrant extract, boysenberries, a boysenberry extract in the manufacture of a medicament or dietary supplement for improving taste, tenderness and/or colour in meat in an animal. Preferably the animal is a bovine animal or a bird.

In another broad aspect, the invention provides the use of blackcurrants, a blackcurrant extract, boysenberries, and/or a boysenberry extract in the manufacture of a medicament or dietary supplement for improving or supporting health of an animal, enhancing or supporting immune system response and/or function, reducing disease mortality, reducing the dependence on antibiotics, assisting or supporting stress recovery, reducing stress impact and/or mortality in confined populations (particularly, for example, in cases of overpopulation, high air temperatures and/or high air humidity), reducing stress impact on meat quality, improving moisture content and tenderness of meat, and/or eliminating unpleasant odours in meat, fat and skin in an animal.

Preferably the animal is a bird or a bovine animal.

In another broad aspect, the invention provides the use of blackcurrants, a blackcurrant extract, boysenberries, and/or a boysenberry extract in the manufacture of a medicament or dietary supplement for improving or supporting egg quality and/or productive egg laying life span, preventing deterioration in egg shell integrity, enhancing egg-laying performance, improving egg quality and colour, improving egg white and yolk texture, increasing polyphenol content, and/or improving nutritional value and/or odour of an egg in a bird. Preferably, the use is for manufacturing a medicament or dietary supplement for improving one or more of shell strength, firmness of egg white, consistency of egg white, vitamin A content, and the number of A grade eggs laid, and maintaining egg shell colour during the laying life of the bird.

In another broad aspect, the invention provides the use of one or more anthocyanins in the manufacture of a medicament or dietary supplement for administration to an animal for preventing or minimising lactic acid production and/or build up during exercise, preventing or minimising exercise-induced muscle damage and/or fatigue, improving or supporting recovery following exercise, reducing heart rate recovery time following exercise, improving stamina and/or endurance, treating and/or preventing a vascular disease, improving taste, tenderness and/or colour of meat, improving or supporting health of an animal, enhancing or supporting immune system response and/or function, reducing disease mortality, reducing the dependence on antibiotics, assisting or supporting stress recovery, reducing stress impact and/or mortality in confined populations (particularly, for example, in cases of overpopulation, high air temperatures and/or high air humidity), reducing stress impact on meat quality, improving moisture content and tenderness of meat, and/or eliminating unpleasant odours in meat, fat and skin, and/or improving or supporting egg quality and/or productive egg laying life span, preventing deterioration in egg shell integrity, enhancing egg-laying performance, improving egg quality and colour, improving egg white and yolk texture, increasing polyphenol content, and/or improving nutritional value and/or odour of an egg in birds. Preferably, where the animal is a hoofed animal the vascular disease is laminitis.

Blackcurrants, a blackcurrant extract, boysenberries, a boysenberry extract, and/or a composition as herein before described for use in preventing or minimising lactic acid production and/or build up during exercise in an animal.

Blackcurrants, a blackcurrant extract, boysenberries, a boysenberry extract, and/or a composition as herein before described for use in preventing or minimising exercise-induced muscle damage and/or fatigue in an animal.

Blackcurrants, a blackcurrant extract, boysenberries, a boysenberry extract, and/or a composition as herein before described for use in improving or supporting recovery following exercise in an animal.

Blackcurrants, a blackcurrant extract, boysenberries, a boysenberry extract, and/or a composition as herein before described for use in reducing heart rate recovery time following exercise in an animal.

Blackcurrants, a blackcurrant extract, boysenberries, a boysenberry extract, and/or a composition as herein before described for use in improving stamina and/or endurance in an animal.

Preferably the animal is a horse, a camel, a bovine animal or a dog.

Blackcurrants, a blackcurrant extract, boysenberries, a boysenberry extract, and/or a composition as herein before described for use in treating and/or preventing a vascular disease in an animal. Preferably, the vascular disease is laminitis. Preferably the animal is a hoofed animal.

Blackcurrants, a blackcurrant extract, boysenberries, a boysenberry extract, and/or a composition as herein before described for use in improving taste, tenderness and/or colour in meat in an animal. Preferably the animal is a bovine animal or a bird.

Blackcurrants, a blackcurrant extract, boysenberries, a boysenberry extract, and/or a composition as herein before described for use in improving or supporting health of an animal, enhancing or supporting immune system response and/or function, reducing disease mortality, reducing the dependence on antibiotics, assisting or supporting stress recovery, reducing stress impact and/or mortality in confined populations (particularly, for example, in cases of overpopulation, high air temperatures and/or high air humidity), reducing stress impact on meat quality, improving moisture content and tenderness of meat, and/or eliminating unpleasant odours in meat, fat and skin in an animal.

Preferably the animal is a bird or a bovine animal.

Blackcurrants, a blackcurrant extract, boysenberries, a boysenberry extract, and/or a composition as herein before described for use in improving or supporting egg quality and/or productive egg laying life span, preventing deterioration in egg shell integrity, enhancing egg-laying performance, improving egg quality and colour, improving egg white and yolk texture, increasing polyphenol content, and/or improving nutritional value and/or odour of an egg in a bird. Preferably, the use is for improving one or more of shell strength, firmness of egg white, consistency of egg white, vitamin A content, and the number of A grade eggs laid, and maintaining egg shell colour during the laying life of the bird.

One or more anthocyanins for use in preventing or minimising lactic acid production and/or build up during exercise, preventing or minimising exercise-induced muscle damage and/or fatigue, improving or supporting recovery following exercise, reducing heart rate recovery time following exercise, improving stamina and/or endurance, treating and/or preventing a vascular disease, improving taste, tenderness and/or colour of meat, improving or supporting health of an animal, enhancing or supporting immune system response and/or function, reducing disease mortality, reducing the dependence on antibiotics, assisting or supporting stress recovery, reducing stress impact and/or mortality in confined populations (particularly, for example, in cases of overpopulation, high air temperatures and/or high air humidity), reducing stress impact on meat quality, improving moisture content and tenderness of meat, and/or eliminating unpleasant odours in meat, fat and skin, and/or improving or supporting egg quality and/or productive egg laying life span, preventing deterioration in egg shell integrity, enhancing egg-laying performance, improving egg quality and colour, improving egg white and yolk texture, increasing polyphenol content, and/or improving nutritional value and/or odour of an egg in birds. Preferably, where the animal is a hoofed animal the vascular disease is laminitis.

In another broad aspect the invention provides a method for preventing or minimising lactic acid production and/or build up during exercise, preventing or minimising exercise-induced muscle damage and/or fatigue, improving or supporting recovery following exercise, reducing heart rate recovery time following exercise, improving stamina and/or endurance, treating and/or preventing a vascular disease, improving taste, tenderness and/or colour of meat, improving or supporting health, enhancing or supporting immune system response and/or function, reducing disease mortality, reducing the dependence on antibiotics, assisting or supporting stress recovery, reducing stress impact and/or mortality in confined populations (particularly, for example, in cases of overpopulation, high air temperatures and/or high air humidity), reducing stress impact on meat quality, improving moisture content and tenderness of meat, eliminating unpleasant odours in meat, fat and skin, improving or supporting egg quality and/or productive egg laying life span, preventing deterioration in egg shell integrity, enhancing egg-laying performance, improving egg quality and colour, improving egg white and yolk texture, increasing polyphenol content, and/or improving nutritional value and/or odour of an egg in an animal, the method comprising administering to the animal one or more anthocyanins or compositions containing one or more anthocyanins. Preferably, where the animal is a hoofed animal the vascular disease is laminitis.

Preferably the anthocyanins are extracted from boysenberries and/or blackcurrants.

Preferably the anthocyanins extracted from boysenberries are selected from the group including: cyanidin-3-glucoside; cyanidin-3-rutinoside; cyanidin-3-spohoroside; cyanidin-3-rutinoside-5-glucoside. Preferably the anthocyanins extracted from blackcurrants are selected from the group including: Delphinidin-3-glucoside; Delphinidin-3-rutinoside; Cyanidin-3-glucoside and Cyanidin-3-rutinoside.

Preferably the flavanoid epigallocateechin is present in extracts from blackcurrants.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1: Reports creatine kinase levels in horses taking a supplement of the present invention versus a control group, pre and post racing a 160 km event.

FIG. 2: Reports aspartate aminotransferase levels in horses taking a supplement of the present invention versus a control group, pre and post racing a 160 km event.

FIG. 3: Reports blood lactate concentrations in horses taking a supplement of the present invention versus a control group, pre and post racing a 160 km event.

FIG. 4: Reports heart rate recovery time in horses taking a supplement of the present invention versus a control group, pre and post racing a 160 km event.

### PREFERRED EMBODIMENT(S)

The following is a description of the present invention, including preferred embodiments thereof, given in general terms. The invention is further elucidated from the disclosure given under the heading “Examples” herein below, which provides experimental data supporting the invention.

The inventors have surprisingly identified that in horses, particularly horses competing in endurance events, compositions containing backcurrant and/or extracts of blackcurrant are of use in aiding in the prevention of muscle damage and fatigue associated with exercise, and also in helping to reduce build up of lactic acid during exercise, and reducing the time for the heart rate to recover to resting rate following exercise. Accordingly, they believe there is value in use of the compositions as dietary supplements to help prevent muscle damage and fatigue associated with exercise, increase stamina and endurance during exercise, and support recovery from exercise (for example, competition and training) and injury. This may help minimise training down-time following a competition or injury, or reduce the effects of intensive training before a competition, which in race horses for example, could have substantial economic benefit.

In addition, whilst the inventors have identified that the dietary supplements have use in preventing exercise-related damage and fatigue and supporting recovery following exercise, and in particular damage, fatigue and recovery associated with endurance events, they also contemplate the use of the supplements to support other areas of animal health. For example, stress recovery, enhanced immune system response, and egg quality and productive egg laying life span (for example, in laying birds, such as chickens). In particular, the inventors have conducted preliminary trials in chickens fed compositions of the invention and have observed significant increases in the quality and quantity of eggs. Of particular note, they have observed an increase in egg quality and the quantity laid, especially in the later stages of the typical laying life (for example, 18 to 24 months old). By way of example, they have observed increased shell strength, improved firmness of the egg white, improved consistency of the egg white, maintenance of shell colour throughout the laying life of the chicken, and approximately 3 times the level of vitamin A of an egg from a hen not taking the supplement. The inventors have observed an increase of up to approximately 4 times the number of first grade eggs laid in chickens (18 to 24 months old) fed the compositions versus those that were not.

In addition, the inventors have found that the dietary supplements have use in improving the taste, tenderness and/or colour of meat. In particular, the inventors have conducted preliminary trials in Angus cattle fed compositions of the invention and have observed improved taste and tenderness in the meat. In addition, the meat of the animals fed compositions of the invention had a bright red colour.

The dietary supplements of the invention were developed following numerous feeding trials in animals. In formulating the compositions the inventors had to overcome a number of significant problems and often competing requirements including dose size, delivery of the desired amount of active ingredients, palatability, shelf life, bioavailability and side-effects.

The following text may describe the invention in terms of dietary supplements for use in horses and/or chickens. However, it should be appreciated that the invention may be applied to a variety of other animals, particularly mammals and birds, including for example, dogs (including, for example, sporting or show dogs, and in particular greyhounds) and camels and bovine animals.

In general terms the invention provides dietary supplements and methods for providing various health benefits to animals. Broadly, the supplements comprise processed whole blackcurrants, one or more natural component extracted from blackcurrants, processed whole boysenberries, and/or one or more natural component extracted from boysenberries. In a preferred embodiment, a supplement of the invention comprises at least processed whole blackcurrants and/or one or more natural component extracted from blackcurrants. In one embodiment, a supplement of the invention comprises processed whole blackcurrants and an extract of blackcurrants. In another preferred embodiment, a supplement comprises a blackcurrant extract and processed whole boysenberries. In another embodiment, a supplement comprises processed whole blackcurrants and processed whole boysenberries.

The ratio of each ingredient in a supplement of the invention may vary depending on such things as the dosage required to be delivered to an animal, the desired volume of the supplement, concentration of anthocyanin(s) present in each ingredient, palatability, and shelf life. With this in mind, and on the basis of the information contained herein, skilled persons will be able to calculate the appropriate ratio of the ingredients of use in the supplements. Further, for certain applications the supplements may contain only one of processed whole blackcurrants, processed whole boysenberries, a blackcurrant extract or a boysenberry extract.

The blackcurrants of use in the invention preferably have an anthocyanin concentration of from approximately 200 mg/100 g of fruit to approximately 400-500 mg/100 g. In a particularly preferred embodiment of the invention, the blackcurrants are chosen from one or more of the following varieties: Ben Ard, Ben Rua and Magnus.

The boysenberries of use in the invention preferably have an anthocyanin concentration of from approximately 100 mg/100 g of fruit to approximately 200 mg/100 g. In a particularly preferred embodiment of the invention, the boysenberries are chosen from one or more of the following varieties: Riwaka, Tasman, Mapua, LDE2 and RS4. Preferably the anthocyanins extracted from boysenberries are selected from the group including: cyanidin-3-glucoside; cyanidin-3-rutinoside; cyanidin-3-spohoroside; cyanidin-3-rutinoside-5-glucoside.

As used herein, “processed whole blackcurrants” are blackcurrants which have been treated in some way with minimal and preferably no removal or loss of any part of the fruit. The blackcurrants are preferably processed in a way that retains a substantial proportion of the activity or benefits of the anthocyanin(s) present therein. The blackcurrants may be dried or pureed, for example.

The phrase “processed whole boysenberries”, as used herein, is intended to refer to boysenberries which have been treated in some way with minimal and preferably no removal or loss of any part of the fruit. The boysenberries are preferably processed in a way that retains a substantial proportion of the activity or benefits of the anthocyanin(s) present therein. The boysenberries may be dried or pureed, for example.

Methodology for processing blackcurrants and boysenberries for use in the invention will be readily appreciated by persons of general skill in the art. However, specific examples are provided herein after.

“Natural components” extracted from blackcurrants and/or boysenberries include individual compounds, such as one or more anthocyanin(s), and more complex mixtures of compounds, tissues and/or fluids contained in the fruit, and includes a blackcurrant or boysenberry extract.

The term “blackcurrant extract” or like terms, as used herein, is intended to refer to a product derived from blackcurrants which has been separated from one or more of the natural components of the whole fruit. The extract may comprise one or more compounds or ingredients and preferably includes one or more anthocyanin(s). The extract may also optionally contain one or more polyphenols and/or vitamin C. The extract is preferably obtained from the skin, pulp and/or seeds of the fruit. Most preferably the extract is obtained from material including the skin. By way of example, in one preferred embodiment, the extract is a blackcurrant pomace and in another preferred embodiment, the extract is an extract obtained from a blackcurrant pomace. As a further example, in an alternative embodiment, the extract is a concentrate of the pressed juice of the fruit.

The term “boysenberry extract”, as used herein, is intended to refer to a product derived from boysenberries which has been separated from one or more of the natural components of the whole fruit. The extract may comprise one or more compounds or ingredients and preferably includes one or more anthocyanin(s). Preferably the anthocyanins extracted from blackcurrants are selected from the group including: Delphinidin-3-glucoside; Delphinidin-3-rutinoside; Cyanidin-3-glucoside and Cyanidin-3-rutinoside. Preferably the flavanoid epigallocateechin is present in extracts from blackcurrants. The extract may also optionally contain one or more polyphenols and/or vitamin C. The extract is preferably obtained from the skin, pulp and/or seeds of the fruit. Most preferably the extract is obtained from material including the skin. By way of example, the extract may be an extract obtained from a boysenberry pomace. As a further example, in an alternative embodiment, the extract is a concentrate of the pressed juice of the fruit.

A blackcurrant or boysenberry extract of use in the invention may be prepared from whole fruit, fresh or frozen, using any appropriate process known in the art. However, exemplary methodology is provided herein after.

A single dose of a supplement of the invention preferably comprises from approximately 1 mg to approximately 1500 mg of anthocyanin(s). In one preferred embodiment a single dose of the composition comprises approximately 1000 mg of anthocyanin(s), which is suitable for a horse. In one preferred embodiment a single dose of the composition comprises approximately 4.5 mg of anthocyanin(s), which is suitable for a chicken.

In a preferred embodiment, a supplement of the invention comprises from approximately 1% to approximately 35% anthocyanins (w/w). In a preferred embodiment, a supplement of the invention comprises from approximately 0.2% to approximately 1% anthocyanins (w/w).

It should be appreciated that a dietary supplement in accordance with the invention may include ingredients in addition to those mentioned herein. For example, they may include fillers, carriers, extenders, flavour enhancers, colourants, compounds which increase shelf life, other fruits including fruit extracts, and/or compounds which may provide additional health benefits. Preferably the supplement also contains flavanoids. In one particularly preferred embodiment the supplement contains the flavanoid epigallocateechin.

Where a supplement of the invention contains more than one ingredient, the various ingredients may be combined or mixed in the desired ratio, using any known methodology.

In use, a supplement of the invention may be administered to an animal in any appropriate way. In one preferred embodiment, a supplement is fed to the animal, preferably by adding it to and mixing it with the animal's normal feed. The supplement may be provided to the animal at any time and frequency. It is preferable that the animal is on a regular maintenance programme of the supplement.

In a preferred embodiment, the animal is fed the supplement regularly to achieve a daily dose of from approximately 0.5 mg/kg of body weight to approximately 4 mg/kg of body weight. In one embodiment, the animal is fed the supplement regularly to achieve a daily dose of approximately 1.5 mg/kg of body weight. Preferably, the supplement is provided to the animal with each of its normal meals. The supplement may be provided in a single dose, or a number of divided doses, to achieve the desired daily dose. The animal may be provided with a single dose mixed with feed, on which it grazes off and on over the course of a 24 hour period. If desired, the supplement may be provided to the animal once every two or more days, rather than daily. In some instances, the dose of the supplement fed to the animal may be increased.

The invention should be considered to encompass a food product, particularly an animal feed, comprising processed whole blackcurrants, a blackcurrant extract, processed whole boysenberries and/or a boysenberry extract, and preferably a dietary supplement as herein described, in combination with one or more food ingredient. In a preferred embodiment, the food product contains from approximately 0.1% to 12.5% of a dietary supplement of the invention of the food product (w/w), which would be suitable for a chicken, for example. In a preferred embodiment, the food product contains from approximately 0.1% to 5% of a dietary supplement of the invention of the food product (w/w), which would be suitable for a horse, for example. The one or more food ingredients may be any food ingredient suitable to consumption by the desired animal. By way of example, the one or more food ingredient may be selected from the group including processed or whole grains, processed or unprocessed vegetable matter, hay, blood and bone or fish meals, molasses and bran or chaff.

The amount of blackcurrant, boysenberry, blackcurrant extract, boysenberry extract, or supplement in the food product may vary dependent on the amount of food to be consumed by the animal, the number of times the animal is to be fed in a particular period, and the desired dose of the supplement (or anthocyanin) required to be given to the animal over a particular period of time. However, by way of example, the animal is fed the supplement regularly to achieve a daily dose of approximately 1.5 mg/kg of body weight.

The invention also encompasses the use of supplements of the invention, blackcurrants, blackcurrant extracts, boysenberries, and/or boysenberry extracts in the manufacture of medicaments and compositions for various purposes as described elsewhere herein. The blackcurrants and/or boysenberries may be processed or unprocessed whole fruit, preferably processed whole fruit.

In addition, the invention encompasses methods for the treatment of animals (as outlined in the “statement of invention” section herein before), the methods comprising administering to an animal blackcurrants, boysenberries, a blackcurrant extract, a boysenberry extract, and/or a supplement of the invention. The blackcurrants and/or boysenberries may be processed or unprocessed whole fruit, preferably processed whole fruit. Appropriate administration or feeding regimes are described elsewhere herein.

The following is a description of a first preferred supplement of the invention. The inventors have found this preferred supplement to be particularly suitable for administration to horses. However, it should be appreciated that it may be applied to other animals, and is particularly appropriate for camels, bovine animals and dogs (including, for example, sporting or show dogs, and in particular greyhounds).

In this embodiment, the dietary supplement comprises a mixture of processed whole blackcurrants and a blackcurrant extract. However, it should be appreciated that the use of processed whole blackcurrants or natural components extracted from blackcurrants alone is envisaged.

Preferably, the whole blackcurrants are processed to provide a seed-in blackcurrant puree. The puree is preferably freeze-dried. Additionally, the freeze-dried puree may be ground (or crushed), more preferably coarsely ground. Preferably, the freeze-dried puree is ground in a manner and to a size which minimises or prevents breaking or rupturing the blackcurrant seeds. By way of example only, the size of the particles in the ground product may be from approximately 400 microns to approximately 4 mm.

The blackcurrant extract in this embodiment is preferably a blackcurrant extract powder. In one preferred embodiment, the blackcurrant extract powder is freeze-dried. The freeze-dried blackcurrant extract powder may preferably be ground, more preferably coarsely ground. In one preferred embodiment, it is ground to a size with is substantially the same as that for the freeze-dried seed-in blackcurrant puree above.

The blackcurrants of use in this embodiment preferably have an anthocyanin concentration of from approximately 200 mg/100 g of fruit to approximately 400-500 mg/100 g.

As mentioned herein before, the ratio of processed whole blackcurrant to blackcurrant extract may vary depending on such things as the dosage required to be delivered to an animal, the desired volume of the supplement, concentration of anthocyanin(s) present in each, palatability, and shelf life. With this in mind, and on the basis of the information contained herein, skilled persons will be able to calculate the appropriate ratio of whole blackcurrant to blackcurrant extract of use in the supplements. For certain applications the supplements may contain only one of processed whole blackcurrants or a blackcurrant extract. In one preferred embodiment, the composition comprises a freeze-dried blackcurrant extract powder and a freeze-dried blackcurrant puree in a ratio of from approximately 3 to 2, to approximately 1 to 24 (w/w). Preferably, the composition comprises a freeze-dried blackcurrant extract powder and a freeze-dried blackcurrant puree in a ratio of approximately 1 to 3 (w/w).

In one preferred embodiment, where the supplement comprises a freeze-dried blackcurrant extract powder and a freeze-dried blackcurrant puree, the freeze-dried extract contains at least approximately 5 times to at least approximately 35 times more anthocyanin compared to freeze-dried blackcurrant puree (w/w). In one embodiment, the freeze-dried extract contains at least approximately 10 times more anthocyanin compared to freeze-dried blackcurrant puree (w/w).

A single dose of a supplement of this embodiment of the invention preferably comprises from approximately 1 mg to approximately 1500 mg of anthocyanin(s). In one preferred embodiment a single does of the composition comprises to approximately 1000 mg of anthocyanin(s).

In a preferred embodiment, a supplement of this embodiment comprises from approximately 1% to approximately 35% anthocyanins (w/w).

Exemplary supplements of this embodiment of the invention include:

| Dosage, Pack Size | Freeze-dried Extract | Freeze-dried Seed-in Puree |
|---|---|---|
| (grams) | Powder (grams) | (grams) |
| 10 | 6 | 4 |
| 20 | 5 | 15 |
| 30 | 4 | 26 |
| 40 | 3 | 37 |
| 50 | 2 | 48 |

where the anthocyanin concentration of each ingredient is as follows:

| | Anthocyanin Conc. | |
|---|---|---|
| | | g of |
| | | powder/puree to |
| | | achieve 1000 mg |
| | mg/100 g | anthocyanin |
| Freeze-dried Extract Powder | 16,000 | 6.25 |
| Freeze-dried Seed-in Puree | 1,382 | 72.36 |

A particularly preferred supplement comprises a dosage of approximately 20 g containing approximately 5 g of the coarsely crushed freeze dried blackcurrant extract powder and approximately 15 g of the coarsely crushed freeze dried seed-in blackcurrant puree.

A blackcurrant puree suitable for use in the invention may be prepared from whole blackcurrants, fresh or frozen, using any appropriate process known in the art, including methods for pureeing, freeze drying and grinding. However, in one embodiment the following methodology is used:
1. Maceration of whole fruit.
2. Direct steam heating and mixing of the fruit at approximately <65° C.
3. Screening through a rectifier with an approximately 0.75 mm porosity screen.
4. Packing into suitable containers and blast frozen at approximately −40° C. for a minimum of approximately 6 hours.
5. Thawing sufficient to place in trays and freeze dry to less than approximately 6% moisture content.
6. Coarsely crushing the product in such a way to prevent breaking or rupturing the blackcurrant seeds.

As mentioned herein before, a blackcurrant extract of use in the invention may be prepared from whole blackcurrants, fresh or frozen, using any appropriate process known in the art. However, in one embodiment, the following methodology is used:
1. Provide a Blackcurrant pomace (the by-product remaining following juice removal) with or without seeds remaining. In the juicing process the blackcurrants may be treated with an enzyme and/or heat to aid juice and sugar removal.
2. Temperature controlled counter-current extraction of the pomace with ethanol or other solvents. Other commercial extraction methods may be used. Ethanolic liquid extract produced nominally contains between approximately 2% to 7% anthocyanin by volume.
3. The extract is evaporated to approximately 10% of the original volume to recover ethanol and prepare the extract for freeze drying.
4. The condensed product is placed in trays and freeze dried to less than approximately 6% moisture content.
5. The freeze dried product is coarsely ground to match the texture of a coarsely crushed freeze dried seed-in blackcurrant puree (as above).

The blackcurrant extract and blackcurrant puree are then mixed in the desired quantities, along with any additional optional ingredients, to form the supplement.

The supplement is preferably packed into containers which provide a gas barrier. In a preferred embodiment the container is a gas barrier foil pouch. The supplement may be packed into the container with or without inert gas flushing to prevent oxidative or photo degradation of the anthocyanin(s). In one embodiment, the supplement is used to improve or support recovery following exercise. In another embodiment, the supplement is used to prevent or minimise muscle damage and/or fatigue during exercise. In another embodiment, the supplement is used to improve stamina and/or endurance. In one particular embodiment, the supplement is used to prevent or minimise lactic acid production and/or build up during exercise and/or reduce the length of time for the heart rate to recover to resting rate following exercise. In another embodiment, the supplement is used to treat and/or prevent a vascular disease. Preferably, where the animal is a hoofed animal the vascular disease is laminitis.

In one particular embodiment, the supplement is used for the above purposes in relation to bouts of exercise for a substantially continuous period of more than 30 minutes, preferably at least approximately 45 minutes, or at least approximately 60 minutes.

In other embodiments, the supplements may be used to: improve taste, tenderness and/or colour of meat; improve or support health; enhance or support the immune system response and/or function; reduce disease mortality, reduce dependence on antibiotics; assist or support stress recovery; reduce stress impact and/or mortality in confined populations (particularly, for example, in cases of overpopulation, high air temperatures and/or high air humidity); improve or support egg quality and/or productive egg laying life span; prevent deterioration in egg shell integrity, enhance egg-laying performance; improve egg shell quality and colour; improve egg white and yolk texture, colour, nutritional value and/or odour; increasing polyphenol content; reduce stress impact on meat quality (such as stress due to pre-slaughter transport); and/or, improve moisture content and tenderness of meat and/or eliminate unpleasant odours in meat, fat and skin. In particular embodiments, the supplements may be used to improve one or more of shell strength, firmness of egg white, egg shell integrity, consistency of egg white, vitamin A content, and the number of A grade eggs laid, and maintaining egg shell colour during the laying life of a bird.

The supplement may be provided to the animal at any time and frequency. It is preferable that the animal is on a regular maintenance programme of the supplement prior to and following bouts of exercise, including training and competition. In a preferred embodiment, the animal is fed the supplement regularly to achieve a daily dose of from approximately 0.5 mg/kg of body weight to approximately 4 mg/kg of body weight. In one embodiment, the animal is fed the supplement regularly to achieve a daily dose of approximately 1.5 mg/kg of body weight. Preferably, the supplement is provided to the animal with each of its normal meals. The supplement may be provided in a single dose, or a number of divided doses, to achieve the desired daily dose. The animal may be provided with a single dose mixed with feed, on which it grazes off and on over the course of a 24 hour period. If desired, the supplement may be provided to the animal once every two or more days, rather than daily. In some instances, for example following bouts of intense or extended periods of exercise, the dose of the supplement fed to the animal may be increased to further aid in recovery. In addition, the dose may be increased (for example to two doses per day) for one or more days in the lead up to an athletic event and for one or more days following such event. The inventors also contemplate administration during an event, where appropriate.

The following provides a description of a second preferred supplement of the invention. The inventors have found this preferred supplement to be particularly suitable for administration to horses. However, it should be appreciated that it may be applied to other animals, and is particularly appropriate for camels, bovine animals and dogs (including, for example, sporting or show dogs, and in particular greyhounds).

In this embodiment, the dietary supplement comprises a mixture of processed whole blackcurrants and a blackcurrant extract.

Preferably, the whole blackcurrants are processed to provide a seed-in blackcurrant puree, preferably a seed-in blackcurrant puree powder. The puree is preferably freeze-dried. Additionally, the freeze-dried puree may be ground (or crushed), more preferably coarsely ground. Preferably, the freeze-dried puree is ground in a manner and to a size which minimises or prevents breaking or rupturing the blackcurrant seeds. By way of example only, the size of the particles in the ground product may be from approximately 400 microns to approximately 4 mm.

The blackcurrant extract in this embodiment is preferably a blackcurrant concentrate, preferably a blackcurrant concentrate powder. Preferably the blackcurrant concentrate powder of formed by grinding a freeze dried blackcurrant concentrate. Use of the blackcurrant concentrate has the advantage of providing vitamin C to the supplement, which may provide added health and therapeutic benefits.

The blackcurrants of use in this embodiment preferably have an anthocyanin concentration as mentioned for the first preferred embodiment. Similarly, the ratio of processed whole blackcurrant to blackcurrant extract may vary as discussed in respect of the first preferred embodiment.

In another preferred embodiment, the composition comprises a blackcurrant concentrate powder and a seed-in blackcurrant puree powder in a ratio of from approximately 3 to 2, to approximately 24 to 1 (w/w). Preferably, the composition comprises a blackcurrant concentrate powder and seed-in blackcurrant puree powder in a ratio of from approximately 3 to 1 (w/w).

In a preferred embodiment, where the supplement comprises a blackcurrant concentrate powder and a seed-in blackcurrant puree powder, the concentrate powder contains at least approximately 1.5 times more anthocyanin compared to seed-in blackcurrant puree powder (w/w). In another preferred embodiment, the concentrate powder contains at least approximately 2 times more anthocyanin compared to seed-in blackcurrant puree powder (w/w).

In this embodiment, a single dose of a supplement of the invention preferably comprises from approximately 1 mg to approximately 1500 mg of anthocyanin(s). In one preferred embodiment a single does of the composition comprises to approximately 1000 mg of anthocyanin(s).

In a preferred embodiment, a supplement of this embodiment comprises from approximately 1% to approximately 35% anthocyanins (w/w).

Exemplary supplements of this embodiment of the invention include:

| Dosage, Pack Size | Freeze-dried Concentrate | Freeze-dried Seed-in Puree |
|---|---|---|
| (grams) | Powder (grams) | (grams) |
| 64 | 20 | 44 |
| 62 | 25 | 37 |
| 59 | 30 | 29 |
| 55 | 40 | 15 |
| 53 | 45 | 8 |

where the anthocyanin concentration of each ingredient is as follows:

| | Anthocyanin Conc. | |
|---|---|---|
| | | g of powder to |
| | | achieve 1000 mg |
| | mg/100 g | anthocyanin |
| Concentrate powder | 2,020 | 49.5 |
| Seed-in puree powder | 1,382 | 72.4 |

A particularly preferred supplement comprises 55g containing 15 g of the seed-in puree powder and 40 g of the concentrate powder.

A blackcurrant seed-in puree of use in this embodiment may be prepared as described in the first preferred embodiment.

A blackcurrant concentrate powder of use in the invention may be prepared from whole blackcurrants, fresh or frozen, using any appropriate process known in the art. However, in one embodiment, the following methodology is used:
1. Milling of whole fruit.
2. Mash heating with or without enzyme addition.
3. Pressing/filtration and removal of pressed pulp and skins.
4. Centrifugation.
5. Filtration.
6. Evaporation.
7. Aseptic filling or non aseptic filling. Where the filling is non aseptic, the product is blast frozen at approximately −40° C. for a minimum of approximately 6 hours.
8. Thawing sufficient to place in trays and freeze dry to less than approximately 6% moisture content.
9. Grinding the final product no finer than 300 microns to prevent dust formation.

The ingredients are then mixed in the desired quantities, along with any additional optional ingredients, to form the supplement.

The supplement is preferably packed into containers as described in relation to the first preferred embodiment.

The supplement of this embodiment may be used for the purposes described in relation to the first preferred embodiment. Similarly, the supplement may be delivered to an animal at any time and frequency, as outlined in relation to the first preferred embodiment.

The following provides a description of a third preferred supplement of the invention. The inventors have found this preferred supplement to be particularly suitable for administration to birds used for meat and egg production (for example, chickens). However, it should be appreciated that it may be applied to other animals.

In one preferred embodiment, the dietary supplement comprises a mixture of a blackcurrant extract and processed whole blackcurrants and/or processed whole boysenberries. However, it should be appreciated that the use of any one of these ingredients alone (or natural components extracted from blackcurrants or boysenberries) is envisaged. In another embodiment, the supplement comprises a mixture of processed whole blackcurrants and processed whole boysenberries.

Preferably, the whole blackcurrants are processed to provide freeze-dried whole blackcurrants. Preferably, the freeze-dried whole blackcurrants are ground (or crushed), more preferably coarsely ground. Preferably, the freeze-dried blackcurrants are ground in a manner and to a size which minimises or prevents breaking or rupturing the blackcurrant seeds. By way of example only, the size of the particles in the ground product may be from approximately 400 microns to approximately 4 mm.

Preferably, the whole boysenberries are processed to provide freeze-dried whole boysenberries. Preferably, the freeze-dried whole boysenberries are ground (or crushed), more preferably coarsely ground. Preferably, the freeze-dried boysenberries are ground in a manner and to a size which minimises or prevents breaking or rupturing the boysenberry seeds. By way of example only, the size of the particles in the ground product may be from approximately 400 microns to approximately 4 mm.

In this embodiment, the blackcurrant extract is preferably a blackcurrant pomace. In a preferred embodiment, the extract is a frozen pomace, more preferably a free-flow frozen or air-dried pomace.

The blackcurrants of use in this embodiment preferably have an anthocyanin concentration of from approximately 80 mg/100 g of fruit to approximately 400-500 mg/100 g. The boysenberries of use in this embodiment preferably have an anthocyanin concentration of from approximately 50 mg/100 g of fruit to approximately 300 mg/100 g.

As mentioned herein before, the ratio of processed whole blackcurrant to blackcurrant extract may vary depending on such things as the dosage required to be delivered to an animal, the desired volume of the supplement, concentration of anthocyanin(s) present in each, palatability, and shelf life. With this in mind, and on the basis of the information contained herein, skilled persons will be able to calculate the appropriate ratio of whole blackcurrant to blackcurrant extract of use in the supplements. For certain applications the supplements may contain only one of processed whole blackcurrants, processed whole boysenberries, or a blackcurrant extract. However, in a preferred embodiment, the composition comprises a frozen blackcurrant pomace (preferably free-flow) and freeze-dried blackcurrants in a ratio of from approximately 1 to 1, to approximately 9 to 1 (w/w). More preferably, the composition comprises a frozen blackcurrant pomace and freeze-dried blackcurrants in a ratio of from approximately 8 to 1 (w/w).

In a preferred embodiment, where the supplement comprises a blackcurrant free-flow frozen pomace and coarsely ground freeze-dried seed-in blackcurrants, the pomace contains at least approximately 2 times to at least approximately 15 times more anthocyanin compared to freeze-dried seed-in blackcurrants (w/w). In one preferred embodiment, the freeze-dried seed-in blackcurrants contains at least approximately 3 times more anthocyanin compared to blackcurrant free-flow frozen pomace (w/w).

In another preferred embodiment, where the supplement comprises a blackcurrant free-flow frozen pomace and coarsely ground freeze-dried seed-in boysenberries, the pomace contains at least approximately 6 times more anthocyanin compared to freeze-dried seed-in boysenberries (w/w). In one embodiment, the pomace contains at least approximately 8 times more anthocyanin compared to freeze-dried seed-in boysenberries (w/w).

In one preferred embodiment, the composition comprises a frozen blackcurrant pomace (preferably free flow) and freeze-dried boysenberries in a ratio of from approximately 1 to 1, to approximately 11 to 1 (w/w). Preferably, the composition comprises a frozen blackcurrant pomace and freeze-dried boysenberries in a ratio of from approximately 9 to 1 (w/w).

A single dose of the supplement preferably comprises from approximately 1 mg to approximately 50 mg of anthocyanin(s). In one preferred embodiment, applying the supplement to chickens, a single dose of the composition comprises to approximately 4.5 mg of anthocyanin(s).

In a preferred embodiment, a supplement of the invention comprises from approximately 1% to approximately 35% anthocyanins (w/w).

Exemplary supplements of a preferred embodiment of the invention include:

| | | | FD Seed-in |
|---|---|---|---|
| Dosage | Blackcurrant | FD Seed-in | Blackcurrant, |
| Size, grams | Pomace, grams | Boysenberry, grams | grams |
| 0.33 | | | 0.33 |
| 1.00 | 1.00 | | |
| 5.63 | | 5.63 | |
| 1.10 | 0.98 | 0.12 | |
| 0.90 | 0.70 | | 0.10 |

where the anthocyanin concentration of each ingredient is as follows:

| | Anthocyanin Conc. | |
|---|---|---|
| | | g of ingredient required to |
| | mg/100 g | achieve 4.5 mg anthocyanin |
| Blackcurrant | 450 | 1.00 |
| Pomace | | |
| FD Seed-in | 80 | 5.63 |
| Boysenberry | | |
| FD Seed-in | 1,382 | 0.33 |
| Blackcurrant | | |

A particularly preferred supplement of this embodiment comprises approximately 1.10 g containing approximately 0.98 g of free-flow blackcurrant pomace and approximately 0.12 g of coarsely crushed freeze-dried seed-in boysenberries.

A blackcurrant free-flow pomace suitable for use in the invention may be prepared from whole blackcurrants, fresh or frozen, using any appropriate process known in the art, including methods for freeze drying and grinding. However, in one embodiment the following methodology is used:
Starting material: blackcurrant pomace, by-product of commercial juice extraction
   1) The pomace is taken fresh from pressing and passed through an IQF freezing system (for example, Octofrost AB) and stored free-flow frozen at less than approximately −18° C.
Or, alternatively:
   1) The pomace is taken fresh from pressing and packed into 20 kg plastic bags that are then placed in wooden bins, static frozen and then stored frozen at less than approximately −18° C.
   2) The frozen pomace is removed from bags and placed through a hammer mill or other size reduction mill with 4 mm screen to make free-flow.
Or, alternatively:
   1) The pomace is taken fresh from pressing or thawed from frozen storage and immediately air-dried to less than approximately 6% moisture content under controlled conditions to maintain anthocyanin content.

Coarsely crushed freeze-dried seed-in blackcurrants may also be prepared using whole blackcurrants, fresh or frozen, using any appropriate process known in the art. However, in one embodiment, the following methodology is used:
Starting material: whole blackcurrants, fresh or frozen
   1) Blackcurrants are thawed (if frozen) sufficient to place in trays and freeze dry to less than approximately 6% moisture content.
   2) Freeze-dried blackcurrants are coarsely crushed in a way to prevent breaking or rupturing the blackcurrant seeds.

Coarsely crushed freeze-dried seed-in boysenberries may also be prepared using whole boysenberries, fresh or frozen, using any appropriate process known in the art. However, in one embodiment, the following methodology is used:
Starting material: whole boysenberries, fresh or frozen
   1) Boysenberries are thawed (if frozen) sufficient to place in trays and freeze dry to less than approximately 6% moisture content.
   2) Freeze-dried boysenberries are coarsely crushed in a way to prevent breaking or rupturing the boysenberries seeds.

The blackcurrant pomace and the coarsely crushed freeze-dried blackcurrants or coarsely crushed freeze-dried boysenberries are then mixed in the desired quantities, along with any additional optional ingredients, to form the supplement. The supplement is preferably packed free-flow frozen, typically into lined commercial cartons. Alternatively, if only air-dried or freeze-dried components are used, the supplement may be packaged in air-tight light barrier packaging and held at room temperature. The cartons or containers in which the supplement is packed preferably provide a gas barrier. The supplement may be packed into the container with or without inert gas flushing to prevent oxidative or photo degradation of the anthocyanin(s).

In one particular embodiment, the supplements are used to: improve or support health; enhance or support the immune system response and/or function; reduce disease mortality, reduce dependence on antibiotics; assist or support stress recovery; reduce stress impact and/or mortality in confined populations (particularly, for example, in cases of overpopulation, high air temperatures and/or high air humidity); reduce stress impact on meat quality (such as stress due to pre-slaughter transport); improve or support egg quality and/or productive egg laying life span; enhance egg-laying performance; improve egg shell quality and colour; prevent deterioration in egg shell integrity, improve egg white and yolk texture, colour, nutritional value and/or odour; increasing polyphenol content; and/or, improve moisture content and tenderness of meat and/or eliminate unpleasant odours in meat, fat and skin. In a preferred embodiment, the supplements are used to improve one or more of shell strength, integrity, firmness of egg white, consistency of egg white, vitamin A content, and the number of A grade eggs laid, and maintaining egg shell colour during the laying life of a bird.

In other embodiments, the supplement may be used for one or more of the following: improving the taste, tenderness and/or colour of the meat of an animal; to improve or support recovery following exercise; to prevent or minimise muscle damage and/or fatigue during exercise; to improve stamina and/or endurance; to prevent or minimise lactic acid production and/or build up during exercise; to reduce the length of time for the heart rate to recover to resting rate following exercise; and/or to treat and/or prevent a vascular disease. Preferably, where the animal is a hoofed animal the vascular disease is laminitis.

The supplement may be provided to the animal at any time and frequency. It is preferable that the animal is on a regular maintenance programme of the supplement. In a preferred embodiment, the animal is fed the supplement regularly to achieve a daily dose of from approximately 1 mg to approximately 20 mg per kg of body weight. In one embodiment, the animal is fed the supplement regularly to achieve a daily dose of approximately 4.5 mg of anthocyanin. Preferably, the supplement is provided to the animal with each of its normal meals. The supplement may be provided in a single dose, or a number of divided doses, to achieve the desired daily dose. The animal may be provided with a single dose mixed with feed, on which it grazes off and on over the course of a 24 hour period. In one embodiment, approximately 8 kg of the supplement is mixed into a total blended feed weight of 1 metric tonne with each hen fed 137 grams of feed per day. If desired, the supplement may be provided to the animal once every two or more days, rather than daily. In addition, the dose may be increased for one or more days to address a particular health issue.

The following provides a description of another preferred supplement of the invention. The inventors have found this preferred supplement to be particularly suitable for administration to beef cattle. However, it should be appreciated that it may be applied to other animals, such as camels or dogs or horses, or particularly other bovine animals, more particularly any one of a number of breeds of bovine animal used for meat production.

In this embodiment, the dietary supplement comprises a mixture of frozen, air-dried or freeze-dried blackcurrant pomace (preferably free flow) and either a freeze-dried blackcurrant extract or frozen, freeze-dried or air-dried blackcurrants

In a preferred embodiment, the composition comprises a frozen, air-dried or freeze-dried blackcurrant pomace (preferably free flow) and a freeze-dried blackcurrant extract powder in a ratio of from approximately 1 to 1, to approximately 100 to 1 (w/w). Preferably, the composition comprises an air-dried blackcurrant pomace and a freeze-dried blackcurrant extract powder in a ratio of approximately 20 to 1 (w/w).

In another preferred embodiment, the composition comprises a frozen, air-dried or freeze-dried blackcurrant pomace (preferably free flow) and frozen, freeze-dried or air-dried blackcurrants in a ratio of from approximately 1 to 1, to approximately 50 to 1 (w/w). Preferably, the composition comprises a frozen blackcurrant pomace and air-dried blackcurrants in a ratio of from approximately 10 to 1 (w/w).

In one embodiment, the supplement may be used for improving the taste, tenderness and/or colour of the meat of an animal. Preferably the animal is a bovine animal. However, it may also be used for any one of the purposes outlined hereinbefore in relation to other preferred supplements of the invention.

### EXAMPLES

The supplements used in these examples contained berry components (blackcurrants) grown in the New Zealand areas Nelson/Tasman and Canterbury, as far south as Timaru. However, there is no specific regional variation in supply for the varieties used.

### Example 1

A study was completed to test the efficacy of the supplement of the invention to aid recovery from exercise induced fatigue and muscle damage associated with competition and training in the equine athlete.

To achieve this, indicators of fatigue and muscle damage were monitored in horses supplemented with the product of the invention verses non-supplemented horses pre and post a trial event of 1601 kms in length. Arguably the 160 km event is one of the most physically stressful and fatiguing competitions of any equine discipline. This distance was utilized in this study to generate the symptoms of fatigue and muscle damage required to test the merits of the product.

### Metabolic and Physiological Parameters

The metabolic and physiological parameters monitored in this trial were blood plasma lactate, creatine kinase, aspartate aminotransferase, and heart rate recovery.

Muscle activity brings about an increased level of circulating lactate in the blood system. Lactate production is a consequence of anaerobic respiration and is recognised as a contributing factor to exercise associated fatigue. Post exercise return to baseline levels of lactate can be used as an index for the capability of recovery. With regards to heart rate, the rate of return of exercise induced elevated heart rate to a pre-exercise resting rate can also be used as an indicator of recovery.

Creatine kinase (CK) is the most sensitive indicator of muscle injury. This enzyme occurs specifically in muscle cells and is a catalyst of ATP synthesis, the energy source for muscle contraction. Elevated levels of CK in the blood system indicate muscle damage or at least increased muscle cell membrane permeability. Levels of serum CK peak within 6 hours of injury but usually return to normal within 2 to 3 days. Aspartate aminotransferase (AST), on the other hand, is not only localized to muscle tissue (for example it is also found in the brain and liver) but it is in muscle tissue that it occurs at its greatest concentration. AST is a key enzyme in amino acid metabolism and forms a useful marker of muscle damage because like CK it is leaked from damaged muscle tissues but in comparison remains detectable in the circulating blood stream for a greater period of time, peaking at approximately 24 hours after damage and may be present at elevated levels for up to five weeks later.

### Methods

8 horses taking part in a 160 km race were monitored. Of these horses 4 were on the supplement while in contrast 4 were kept as controls and received no supplement. Of the 4 supplemented horses 2 of these horses received the supplement at a rate of 70 g per day over the 4 weeks preceding the race with the quantity being increased to 100 g per day for the three days preceding the race and following the race. For the remaining two supplemented horses supplement feeding commenced one week prior to the race at a rate of 100 g per day. These horses also continued to be supplemented post race.

The supplement used in the trial comprised a freeze-dried black current puree which was mixed with the animals normal feed. The supplement contained approximately 1000 mg of anthocyanins per 72 g supplement, or 1382 mg/100 g.

Blood samples were obtained from the horses and AST, CK, and lactate levels were measured using standard procedures by a commercial laboratory in Hamilton, New Zealand. Heart rate was monitored using standard procedures.

### Results

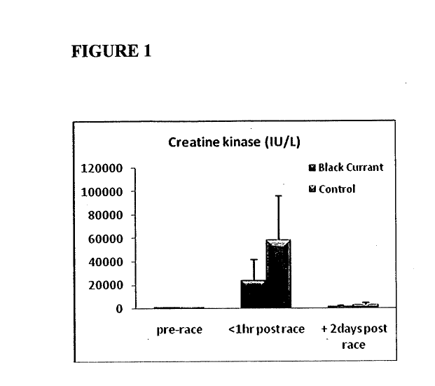

The results indicate several interesting trends support a positive role for the supplement in both aiding competitive performance and supporting post exercise recovery. These results are provided in FIGS. 1 to 4.

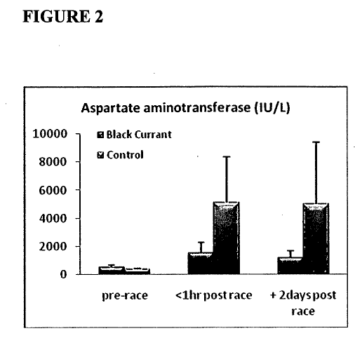

With regards to blood CK concentrations, elevated levels were measured in the samples collected from both supplemented and non-supplemented horses within an hour of race completion (<1 hr post race). In comparison, the non-supplemented group had an elevated blood CK level approximately 3 times higher than that of the supplemented group, indicating a greater degree of muscle damage or permeability within the non-supplemented horses (FIG. 1). This result is further supported by the results obtained for the second monitored muscle enzyme, AST, which as indicated in FIG. 2 shows a post race elevation in the non-supplemented group approximately 3 times higher in concentration than AST levels within the supplemented group.

By two days post race, the CK levels within both groups had fallen back towards the pre-race baseline (FIG. 1) while in contrast AST levels continue to remain elevated (FIG. 2). This result reflects the different retention periods of the enzymes in the blood system with CK levels known to peak within 6 hrs post injury and fall back to base levels within 2 to 3 days, while AST levels are expected to peak approximately 24 hrs post muscle damage and linger at elevated levels for a number of weeks.

Large standard errors within the data of the non-supplemented horses, indicates a broader variability of response with in this group with regards to the measured parameters CK and AST. This may be influenced by a number of factors such as differing levels of fitness between horses. The inventors suggest that the less apparent variability in the supplemented horses, to a degree, may have been overcome with supplementation.

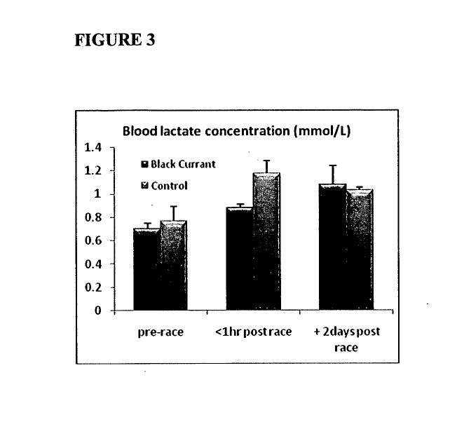

Measurements of blood lactate levels indicate a significant difference in lactate concentration between the supplemented horses and the non-supplemented group at completion of the race (sample period<1 hr post race, FIG. 3). This difference, of approximately 25% less lactate production by the supplemented horses, suggests that over the course of the race the supplemented horses either generated less lactic acid or conversely were able to clear lactate faster from their bloodstream in comparison to the non-supplemented group. The data indicates that there was no significant difference between the two groups with regard to blood lactate levels prior to the race (pre-race) or 2 days post the race (+2 days post race), with lactate levels remaining elevated for both groups at the two day post race sampling period. These lactate results suggest a positive effect for the supplement during the race or at least within an hour of race completion.

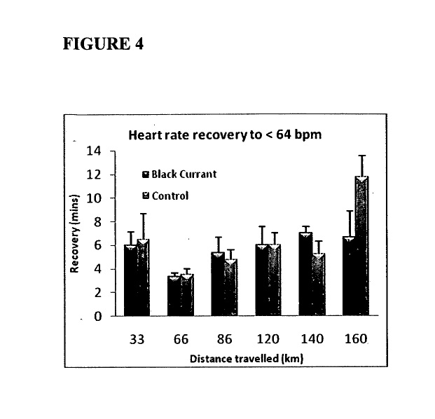

Heart rate recovery data also indicates a supplement induced response (FIG. 4). This response however is not evident until the end of the 160 km distance. Throughout most of the race the average time period for each individual horse's heart rate to drop below the mandatory resting 64 bpm was between 3 and 7 minutes, this was similar for both supplemented and non-supplemented horses (FIG. 4). At the 160 km mark however, the average time period for the non-supplemented horse's heart rate to drop below 64 bpm had increased to approximately 12 minutes, while in contrast the BC supplemented horses recovery rate remained at approximately 7 minutes. This result, when viewed with respect to the lactate results, suggests that at the completion of the race the non-supplemented horses were suffering from a greater degree of fatigue indicated by higher levels of circulating lactate and a slower rate of heart rate recovery with respect to their supplemented counterparts. The AST and CK results also indicate that this group suffered from a higher degree of race induced muscle damage.

The results suggest a supplement of the invention has efficacy as an aid in the prevention of muscle damage and fatigue associated with training, as well as a support for recovery from competition and injury. Results also support use of the supplement of the invention in helping to reduce lactic acid build up during exercise, which is essential for boosting stamina and performance. In particular the results demonstrate the efficacy of the supplement for the above purposes in relation to bouts of intense exercise and/or prolonged periods of exercise, such as is the case in endurance events. The use of the supplement in reducing heart rate recovery time following exercise has benefit in reducing stress on the heart. In addition, in equestrian endurance events, horses' heart rates are monitored at key stages in the event. The horse must stop and have its heart rate checked and is not able to start racing again until its heart rate recovers to a base level. During this time, the horse is still on the clock. A supplement of the invention may help reduce the time lost during these check points in the race.

Whilst the results demonstrate efficacy in horses, the inventors believe the supplement may be of similar use in other animals.

It is of advantage that the supplement contains no prohibited substances and thus may be legally used in various sporting and commercial arenas, for example in race and eventing horses.

### Example 2

### Study 1

A study was completed to test the efficacy of the product on laying hens to improve the laying rates and to reduce the death rate.

### Method

Under closely controlled conditions of a commercial egg farm in Japan, a population of approximately 1,600 laying hens were fed the supplement in the preferred form equivalent to approximately 0.98 grams of blackcurrant pomace and 0.12 grams of freeze-dried seed-in boysenberry providing approximately 4.5 mg per dose per day per hen. To achieve this in a practical sense, 6.9 kg of the supplement was mixed into a final volume of 960 kg of chicken feed. The supplement therefore made up 0.7% of the total chicken feed. The average feed consumption per hen was 137 grams per day. Feeding with the prescribed dose of supplement was carried out daily for 4.5 months during the summer season that causes elevated stress on the chickens.

### Results

TABLE 1
No Pomace Feeding:

| | Chicken | Eggs | Laying | Chicken | Death |
|---|---|---|---|---|---|
| 2008 | Number | Produced | Ratio | Deaths | Ratio |
| June | 1688 | 22173 | 74.1% | 50 | 5.0% |
| July | 2289 | 21749 | 58.7% | 79 | 6.6% |
| August | 2255 | 23870 | 65.8% | 33 | 2.8% |
| September | 2129 | 23288 | 70.1% | 22 | 2.0% |

Laying rate theoretical 100%=0.591 eggs/day/chicken

Ambient conditions worse in 2009—temperature and humidity

TABLE 2
Pomance Feeding Commenced mid-May 2009:

| | Chicken | Eggs | Laying | Chicken | Death |
|---|---|---|---|---|---|
| 2009 | Number | Produced | Ratio | Deaths | Ratio |
| June | 1575 | 30176 | 75.6% | 11 | 0.8% |
| July | 1566 | 29828 | 73.1% | 4 | 0.3% |
| August | 1605 | 26545 | 77.4% | 4 | 0.4% |
| September | 1595 | 25703 | 78.0% | 11 | 1.0% |

The results indicate a positive support role for the supplement in both aiding laying rates and reducing death rates.

As indicated in Tables 1 and 2, death rates were reduced greatly through the summer season in the hens fed the supplement and there were significantly increased laying rates in the hens fed the supplement as compared to the same time period in the previous year.

The supplement was found to reduce the stress related conditions and reduced the summer death rate in the laying chicken population from 3-9 per 1,000 per week to less than 2 per 1,000 per week.

The average temperature and humidity were actually both higher during the summer when the supplement was fed than in the previous year. Higher temperatures and humidity generally have a negative impact on laying rates and death rates.

### Study 2

A further study was carried out in New Zealand with a laying population of 30 hens to determine efficacy of the product in improving egg quality. General analyses were performed on composite egg samples before and after supplementation.

### Method

A laying population of 30 hens in New Zealand were fed the supplement in the preferred form equivalent to approximately 0.98 grams of blackcurrant pomace and 0.12 grams of freeze-dried seed-in boysenberry providing approximately 4.5 mg per dose per day per hen. To achieve this in a practical sense, 6.9 kg of the supplement was mixed into a final volume of 960 kg of chicken feed. The supplement therefore made up 0.7% of the total chicken feed. The average feed consumption per hen was 137 grams per day. Feeding with the prescribed dose of supplement was carried out daily for 4 weeks.

Composite egg samples were analysed and the fat content, anthocyanin content, cholesterol content, total phenolics content and vitamin A (as retinol) content were all assessed.

### Results

TABLE 3
Egg Analysis Comparison

| Analysis | Before | After |
|---|---|---|
| Total Fat (g/100 g) | 1.2 | 0.5 |
| Anthocyanin (mg/100 g) | <1 | 1 |
| Cholesterol (mg/100 g) | 354 | 501 |
| Total Phenolics (mg/100 g) | 150 | 500 |
| Vitamin A (as retinol) (mg/100 g) | 2.97 | <1 |

Sample Analyzed=Whole Eggs—Composite of 4 removed from shell

Laying population=30 hens of the same age and breeding

Avian supplement feeding @ 4.5 mg anthocyanin/day per hen for 4 weeks

The results indicate a positive support role for the supplement in improving egg quality.

As shown in Table 3, there was an over three-fold increase in total phenolics following supplementation for 4 weeks. The inventors believe this may prove to enhance antioxidant capacity of the egg.

It is also positive that the supplement had minimal effect on the anthocyanin content in the actual egg.

### Physical Differences

Observation of the physical differences in egg quality included much firmer egg whites, slightly darker yolk colour and reduced raw and cooked egg odour in the eggs following supplementation.

Whilst the results demonstrate efficacy in chickens, the inventors believe the supplement may be of similar use in other animals.

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

Titles, headings, or the like are provided to enhance the reader's comprehension of this document, and should not be read as limiting the scope of the present invention.

The entire disclosures of all applications, patents and publications, cited above and below, if any, are hereby incorporated by reference. However, the reference to any applications, patents and publications in this specification is not, and should not be taken as, an acknowledgment or any form of suggestion that they constitute valid prior art or form part of the common general knowledge in any country in the world.

Throughout this specification, unless the context requires otherwise, the words “comprise”, “comprising” and the like, are to be construed in an inclusive sense as opposed to an exclusive sense, that is to say, in the sense of “including, but not limited to”.

1-122. (canceled)

123. A composition comprising whole blackcurrants and an extract of blackcurrants, wherein the ratio of extract to whole blackcurrants is from approximately 1 to 24 to approximately 50 to 1.

124. The composition as claimed in claim 123 wherein the anthocyanin concentration is from approximately 0.2% to approximately 35% (w/w).

125. The composition as claimed in claim 123 wherein the whole blackcurrants and/or the extract are dried, preferably freeze-dried.

126. The composition as claimed in claim 123 wherein the whole blackcurrants and/or the extract are frozen.

127. The composition as claimed in claim 123 wherein the whole blackcurrants and/or the extract are ground, preferably coarsely ground.

128. The composition as claimed in claim 123 wherein the whole blackcurrants comprise a seed-in blackcurrant puree.

129. The composition as claimed in claim 123 wherein the blackcurrant extract is an extract of the skin, seeds and/or pulp of the blackcurrant (preferably a pomace), or a blackcurrant concentrate (preferably a blackcurrant concentrate powder).

130. The composition of claim 123 wherein the composition is chosen from the group consisting of:
a. a composition comprising a freeze-dried blackcurrant extract powder and a freeze-dried blackcurrant puree in a ratio of from approximately 3 to 2 to approximately 1 to 24 (w/w), preferably approximately 3 to 1 (w/w);
b. a composition comprising a blackcurrant concentrate powder and a seed-in blackcurrant puree powder in a ratio of from approximately 3 to 2 to approximately 24 to 1 (w/w), preferably approximately 3 to 1 (w/w);
c. a composition comprising a frozen blackcurrant pomace and freeze-dried blackcurrants in a ratio of from approximately 1 to 1 to approximately 9 to 1 (w/w), preferably approximately 8 to 1 (w/w);
d. a composition comprising a frozen, air-dried or freeze-dried blackcurrant pomace (preferably free flow) and frozen, freeze-dried or air-dried blackcurrants in a ratio of from approximately 1 to 1 to approximately 50 to 1 (w/w), preferably approximately 10 to 1 (w/w).

131. The composition of claim 123 wherein a single dose of the composition comprises from approximately 1 mg to approximately 1500 mg anthocyanin(s), preferably approximately 1 mg to approximately 50 mg, approximately 1 mg to approximately 15 mg, approximately 800 mg to approximately 1500 mg, approximately 100 mg, or approximately 4.5 mg.

132. An animal feed comprising a composition of claim 123.

133. The animal feed as claimed in claim 132 wherein the animal feed comprises approximately 0.1% to approximately 12.5% (w/w) of the composition, preferably approximately 0.1% to approximately 5%.

134. The animal feed of claim 132 wherein the animal feed has an anthocyanin content per kg of feed of from approximately 2,000 mg to approximately 60,000 mg of anthocyanin(s), preferably approximately 50000 mg or approximately 4000 mg.

135. A method for preventing or minimising lactic acid production and/or build up in a non-human animal during exercise, for preventing or minimising exercise-induced muscle damage and/or fatigue in a non-human animal, for improving or supporting recovery of a non-human animal following exercise, for reducing heart rate recovery time in a non-human animal following exercise, and/or for improving stamina and/or endurance in a non-human animal the method comprising administering to the animal:
a composition comprising whole blackcurrants and an extract of blackcurrants, wherein the ratio of extract to whole blackcurrants is from approximately 1 to 24 to approximately 50 to 1, or,
an animal feed comprising said composition.

136. The method of claim 135 wherein the animal is a horse, a camel, a bovine animal, or a dog.

137. A method for treating and/or preventing a vascular disease in a non-human animal, the method comprising administering to the animal:
a composition comprising whole blackcurrants and an extract of blackcurrants, wherein the ratio of extract to whole blackcurrants is from approximately 1 to 24 to approximately 50 to 1, or,
an animal feed comprising said composition.

138. The method of claim 137 wherein the vascular disease is laminitis and the animal is preferably a hoofed animal.

139. A method for improving taste, tenderness and/or colour in meat in a non-human animal, the method comprising administering to the animal:
a composition comprising whole blackcurrants and an extract of blackcurrants, wherein the ratio of extract to whole blackcurrants is from approximately 1 to 24 to approximately 50 to 1, or,
an animal feed comprising said composition.

140. A method for improving or supporting health of a non-human animal, enhancing or supporting immune system response and/or function, reducing disease mortality, reducing the dependence on antibiotics, assisting or supporting stress recovery, reducing stress impact and/or mortality in confined populations (particularly, for example, in cases of overpopulation, high air temperatures and/or high air humidity), reducing stress impact on meat quality, improving moisture content and tenderness of meat, and/or eliminating unpleasant odours in meat, fat and skin in an animal, the method comprising administering to the animal:
a composition comprising whole blackcurrants and an extract of blackcurrants, wherein the ratio of extract to whole blackcurrants is from approximately 1 to 24 to approximately 50 to 1, or,
an animal feed comprising said composition.

141. The method of claims 139 wherein the animal is a bird or a bovine animal.

142. The method of claims 140 wherein the animal is a bird or a bovine animal.

143. The method of any one of claims 135 wherein the composition or feed is administered to the animal in sufficient quantity to provide a dose of approximately 0.5 to approximately 20 mg of anthocyanin(s) per kg of the animal per day, preferably approximately 0.5 to approximately 5 mg/kg/day, approximately 4.5 mg/kg/day or approximately 1.5 mg/kg/day.

144. A method for improving or supporting in a bird egg quality and/or productive egg laying life span, preventing deterioration in egg shell integrity, enhancing egg-laying performance, egg quality and colour, egg white and yolk texture, nutritional value and/or odour, increasing polyphenol content in an egg, improving one or more of shell strength, shell integrity, firmness of egg white, consistency of egg white, vitamin A content, the number of A grade eggs laid and maintaining egg shell colour during the laying life of the bird, the method comprising administering to the bird blackcurrants or a composition comprising one or more of processed whole blackcurrants, processed whole boysenberries, one or more natural component extracted from blackcurrants, and one or more natural component extracted from boysenberries.

145. The method of claim 144 wherein the composition comprises a mixture of processed whole blackcurrants and a blackcurrant extract, or a blackcurrant extract and processed whole boysenberries, or processed whole blackcurrants and processed whole boysenberries.

146. The method of claim 145 wherein the composition is chosen from the group consisting of:
a. A composition in which the extract is in the form of a freeze-dried blackcurrant extract powder and the processed whole blackcurrants are in the form of a freeze-dried blackcurrant puree and are present in a ratio of from approximately 3 to 2, to approximately 1 to 24 (w/w), preferably approximately 1 to 3;
b. a composition in which the extract is in the form of a blackcurrant concentrate powder and the processed whole blackcurrants are in the form of a seed-in blackcurrant puree powder and are present in a ratio of from approximately 3 to 2, to approximately 24 to 1 (w/w), preferably approximately 3 to 1;
c. a composition in which the extract is in the form of a frozen blackcurrant pomace and the processed whole blackcurrants are in the form of freeze-dried blackcurrants and are present in a ratio of from approximately 1 to 1, to approximately 9 to 1 (w/w), preferably approximately 8 to 1; and,
d. a composition in which the blackcurrant extract is in the form of a frozen blackcurrant pomace and the processed whole boysenberries are in the form of freeze-dried boysenberries and are present in a ratio of from approximately 1 to 1, to approximately 11 to 1 (w/w), preferably approximately 9 to 1.

147. The method of claim 144 wherein the composition comprises a frozen, air-dried or freeze-dried blackcurrant pomace and a freeze-dried blackcurrant extract powder in a ratio of from approximately 1 to 1 to approximately 100 to 1 (w/w), preferably approximately 20 to 1.

148. The method of claim 144 wherein the composition comprises a frozen, air-dried or freeze-dried blackcurrant pomace and frozen, freeze-dried or air-dried blackcurrants in a ratio of from approximately 1 to 1 to approximately 50 to 1 (w/w), preferably approximately 10 to 1.

149. The method of claim 144 wherein the composition comprises from approximately 0.2% to approximately 35% anthocyanins (w/w).

150. The method as claimed in claim 144 wherein a single dose of the composition comprises from approximately 1 mg to approximately 1500 mg anthocyanin(s), preferably approximately 1 mg to approximately 50 mg anthocyanin(s), or approximately 4.5 mg anthocyanin(s).

151. A method as claimed in claim 144 wherein the blackcurrants, blackcurrant extract, boysenberries, boysenberry extract, and/or the composition are administered to the bird in sufficient quantity to provide a dose of approximately 0.5 to approximately 5 mg of anthocyanin(s) per kg of the bird per day, preferably approximately 1.5 mg/kg/day.

152. A composition comprising processed whole boysenberries and an extract of blackcurrants, wherein the ratio of extract to processed whole boysenberries is from approximately 1 to 1 to approximately 11 to 1.

153. An animal feed comprising a composition as claimed in claim 152.

154. The method of any one of claims 137 wherein the composition or feed is administered to the animal in sufficient quantity to provide a dose of approximately 0.5 to approximately 20 mg of anthocyanin(s) per kg of the animal per day, preferably approximately 0.5 to approximately 5 mg/kg/day, approximately 4.5 mg/kg/day or approximately 1.5 mg/kg/day.

155. The method of any one of claims 139 wherein the composition or feed is administered to the animal in sufficient quantity to provide a dose of approximately 0.5 to approximately 20 mg of anthocyanin(s) per kg of the animal per day, preferably approximately 0.5 to approximately 5 mg/kg/day, approximately 4.5 mg/kg/day or approximately 1.5 mg/kg/day.

156. The method of any one of claims 140 wherein the composition or feed is administered to the animal in sufficient quantity to provide a dose of approximately 0.5 to approximately 20 mg of anthocyanin(s) per kg of the animal per day, preferably approximately 0.5 to approximately 5 mg/kg/day, approximately 4.5 mg/kg/day or approximately 1.5 mg/kg/day.
